(19) United States
(12) Patent Application Publication
(10) Pub. No.: US 2026/0363595 A1
(43) Pub. Date: Nov. 19, 2026

(54) Method and Apparatus for Proposing Arrangement for Electromagnetic Sensor, and Method for Estimating Location of Current Source

(71) Applicants: SHIMADZU CORPORATION, Kyoto-shi, Kyoto (JP); RIKEN, Wako-shi, Saitama (JP)

(72) Inventors: Tomohiro GOMI, Kyoto-shi, Kyoto (JP); Sadamu TOMITA, Kyoto-shi, Kyoto (JP); Ryu UMEBAYASHI, Kyoto-shi, Kyoto (JP); Okito YAMASHITA, Wako-shi, Saitama (JP); Yusuke TAKEDA, Wako-shi, Saitama (JP)

(73) Assignees: SHIMADZU CORPORATION, Kyoto-shi, Kyoto (JP); RIKEN, Wako-shi, Saitama (JP)

(21) Appl. No.: 19/118,316

(22) PCT Filed: Oct. 2, 2023
(86) PCT No.: PCT/JP2023/035835

§ 371 Date: Apr. 3, 2025

(30) Foreign Application Priority Data
Oct. 5, 2022 (JP) 2022-160978

(51) Int. Cl. A61B 5/242

(52) U.S. Cl. CPC A61B 5/242

(57) ABSTRACT

A method for proposing an arrangement for an electromagnetic sensor is a method for proposing an arrangement for a plurality of electromagnetic sensors on a biological surface around a target region inside a subject, the method using a forward model for estimating an electromagnetic field generated at positions of the plurality of electromagnetic sensors by a biological activity caused inside the subject, the method comprising: setting a calculation condition including a location of the target region and available positions for arranging the plurality of electromagnetic sensors; calculating a proposed arrangement based on the set calculation condition for all or some of the plurality of electromagnetic sensors for estimating a location of a current source inside the subject; and outputting the proposed arrangement.

### TECHNICAL FIELD

The present disclosure relates to a method and apparatus for proposing an arrangement for an electromagnetic sensor, and a method for estimating a location of a current source.

### BACKGROUND ART

A technique using a plurality of electromagnetic sensors arranged on a surface of the living body of a subject to detect a biological activity caused inside the living body is known. For example, in magnetoencephalography (MEG), a plurality of sensors arranged on the head of a subject can detect brain magnetism caused by neural activity. In order to clarify brain function, it is desirable to accurately estimate the location of a brain activity site or a current source (hereinafter, also referred to as “current source estimation”). One approach to improve accuracy of current source estimation is to measure brain magnetism with high sensitivity. In order to measure brain magnetism with high sensitivity, a positional relationship between a sensor and the head is important. Specifically, it is important to bring the sensor close to the brain activity site and to arrange the sensor at an appropriate position in accordance with the brain activity.

Conventional magnetoencephalography uses a plurality of superconducting quantum interference devices (SQUIDs) sensors each requiring cooling with liquid helium. Each SQUID sensor is disposed in a dewar filled with liquid helium and positionally fixed and thus arranged.

In contrast, small magnetic sensors that operate at room temperature, such as an OPM (optically pumped magnetometer) sensor, a TMR (tunnel magneto resistance) sensor, and an NVC (nitrogen-vacancy center in diamond) sensor, have been actively developed. These sensors do not require cooling with liquid helium and is positionally changeable, and it is possible to easily change arrangement of the sensors in accordance with a brain activity site, which has been difficult in conventional art. For example, Japanese Patent Laying-Open No. 2020-151023 discloses that the location of a Broadmann area of the head of a subject is determined from an MRI (magnetic resonance imaging) image of the head of the subject, and a single OPM sensor is disposed on a surface of the head close to the determined Broadmann area.

### CITATION LIST

### Patent Literature

PTL 1: Japanese Patent Laying-Open No. 2020-151023

### SUMMARY OF INVENTION

### Technical Problem

When an OPM sensor or a similar small electromagnetic sensor that operates at room temperature is used to perform current source estimation, it is desirable to dispose a plurality of such sensors, rather than a single such sensor, on a biological surface, as well as when a conventional SQUID is used to do so. And when the number of sensors is limited for some reason, it is desirable to dispose the limited number of sensors at appropriate positions. However, Japanese Patent Laying-Open No. 2020-151023 does not indicate any appropriate arrangement for a plurality of sensors.

Furthermore, even when the arrangement of each sensor is fixed as it is for a SQUID, which output of a plurality of sensors is to be used has been insufficiently studied conventionally.

The present disclosure has been made to solve the above problem, and one object of the present disclosure is to propose an arrangement for a plurality of sensors suitable for current source estimation. Another object of the present disclosure is to provide a method for optimally utilizing outputs of the plurality of sensors.

### Solution to Problem

According to the present disclosure, a method for proposing an arrangement for an electromagnetic sensor is a method for proposing an arrangement for a plurality of electromagnetic sensors on a biological surface around a target region inside a subject, the method using a forward model for estimating an electromagnetic field generated at positions of the plurality of electromagnetic sensors by a biological activity caused inside the subject, the method comprising: setting a calculation condition including a location of the target region and available positions for arranging the plurality of electromagnetic sensors; calculating a proposed arrangement based on the set calculation condition for all or some of the plurality of electromagnetic sensors for estimating a location of a current source inside the subject; and outputting the proposed arrangement.

According to the present disclosure, a method for estimating a location of a current source is a method for estimating a location of a current source inside a subject by using a result of detection by a plurality of electromagnetic sensors each disposed on a biological surface of the subject to detect an electromagnetic field generated by a biological activity caused in a target region inside the subject, the method comprising: setting a calculation condition including a location of the target region and an arrangement for the plurality of electromagnetic sensors; calculating priority based on the calculation condition for all or some of the plurality of electromagnetic sensors; and estimating the location of the current source using a result of detection by an electromagnetic sensor selected based on the priority.

According to the present disclosure, an apparatus that proposes an arrangement for an electromagnetic sensor is an apparatus that proposes an arrangement for a plurality of electromagnetic sensors on a biological surface around a target region inside a subject, the apparatus using a forward model for estimating an electromagnetic field generated at positions of the plurality of electromagnetic sensors by a biological activity caused inside the subject, the apparatus comprising: an input unit that receives a calculation condition including a location of the target region and available positions for arranging the plurality of electromagnetic sensors; a computing device that calculates a proposed arrangement based on the calculation condition for all or some of the plurality of electromagnetic sensors for estimating a location of a current source inside the subject; and an output unit that outputs the proposed arrangement.

### Advantageous Effects of Invention

The presently disclosed arrangement proposing method and apparatus can propose an arrangement for a plurality of sensors suitable for current source estimation.

The presently disclosed location estimating method can accurately estimate the location of a current source by using a result of detection by a sensor having higher priority selected from a plurality of electromagnetic sensors.

### BRIEF DESCRIPTION OF DRAWINGS

FIG. 1 schematically, generally shows a configuration of a data processing system used in a method for proposing an arrangement for an electromagnetic sensor.

FIG. 2 is a diagram for illustrating a concept of a method for proposing an arrangement for an OPM sensor.

FIG. 3 is a flowchart of an example of a procedure of an arrangement proposing process.

FIG. 4 is a diagram showing an example of arranging sensors when 11 OPM sensors are used to perform magnetoencephalographic measurement twice, and a result of the measurement.

FIG. 5 is a diagram comparing in accuracy a measurement for a first time (for a uniform arrangement) and a measurement for a second time (for an optimal arrangement) shown in FIG. 4.

### DESCRIPTION OF EMBODIMENTS

Hereinafter, embodiments of the present disclosure will be described in detail with reference to the drawings. In the drawings, identical and equivalent components are identically denoted and will not be described repeatedly.

### First Embodiment

### <System Configuration>

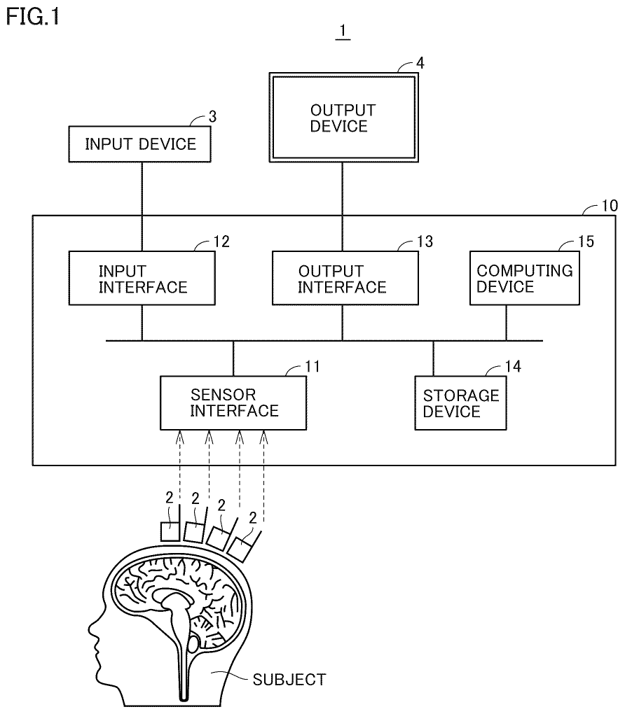

FIG. 1 schematically, generally shows a configuration of a data processing system 1 used in a method for proposing an arrangement for an electromagnetic sensor according to the present embodiment. In the present embodiment, as an example of the electromagnetic sensor, an OPM sensor 2, which is a small magnetic sensor operating at room temperature, will be described.

Data processing system 1 comprises a plurality of OPM sensors 2, an input device 3, an output device 4, and a processing device 10. Data processing system 1 is configured to use results of detection by the plurality of OPM sensors 2 to estimate the location of a biological activity site (or a current source) in the brain of a subject.

The plurality of OPM sensors 2 are each arranged on a surface of the head of the subject by a user (medical personnel or the like) to detect magnetism generated at the position of the arranged sensor by a biological activity caused in the brain of the subject. The plurality of OPM sensors 2 may or may not be in direct contact with the surface of the head of the subject. For example, a cover member for holding sensor 2 may be attached to the head of the subject. FIG. 1 shows an example with four OPM sensors 2 arranged on the surface of the head of the subject.

Each OPM sensor 2 operates at room temperature, and does not need to be disposed in a dewar as a SQUID does as described above. The plurality of OPM sensors 2 can thus be arranged variably depending on the brain activity.

Input device 3 is for example a keyboard or a mouse or a similar pointing device and receives information (e.g., a calculation condition described hereinafter) input by a user. The information input to input device 3 is sent to processing device 10. Output device 4 includes, for example, a liquid crystal display (LCD) panel, and it is a display that displays information to a user.

Processing device 10 includes, as main hardware components, a sensor interface 11, an input interface 12, an output interface 13, a storage device 14, and a computing device 15. Processing device 10 may be implemented for example as a general-purpose computer, or may be implemented by a computer (a server or the like) dedicated to data processing system 1.

Sensor interface 11 is an interface for interconnecting processing device 10 and the plurality of OPM sensors 2, and implements input and output of signals between processing device 10 and the plurality of OPM sensors 2. Input interface 12 is an interface for interconnecting processing device 10 and input device 3 and implements input and output of signals between processing device 10 and input device 3. Output interface 13 is an interface for interconnecting processing device 10 and output device 4 and implements input and output of data between processing device 10 and output device 4.

Storage device 14 stores information (a program or the like) used for processing by computing device 15. Note that input information (e.g., a calculation condition described hereinafter) input by the user to input device 3 is stored in storage device 14.

Computing device 15 includes a CPU (a central processing unit), and uses the information stored in storage device 14 and a result of measurement by the plurality of OPM sensors 2 to perform “current source estimation” to estimate the location of a current source (or a biological activity site) in the brain of the subject.

Computing device 15 causes output device 4 to display the location of the current source determined through the current source estimation as the biological activity site. The user can see the location of the biological activity site in the brain of the subject by looking at contents displayed on output device 4.

### <Proposing Arrangement for Sensors Suitable for Current Source Estimation>

In order to accurately estimate the location of a current source in a brain, it is desirable to measure the brain's magnetism with high sensitivity using a plurality of OPM sensors 2. In order to measure the brain's magnetism with high sensitivity, a positional relationship between the plurality of OPM sensors 2 and the current source in the brain is important. Specifically, it is important to arrange the plurality of OPM sensors 2 at appropriate positions in accordance with the location of the current source in the brain.

According to the present embodiment, computing device 15 is configured to perform a process for proposing an arrangement for OPM sensors 2 suitable for current source estimation (hereinafter also referred to as an “arrangement proposing process”). Hereinafter, an arrangement proposed for OPM sensors 2 through the arrangement proposing process will also simply be referred to as a “proposed arrangement”.

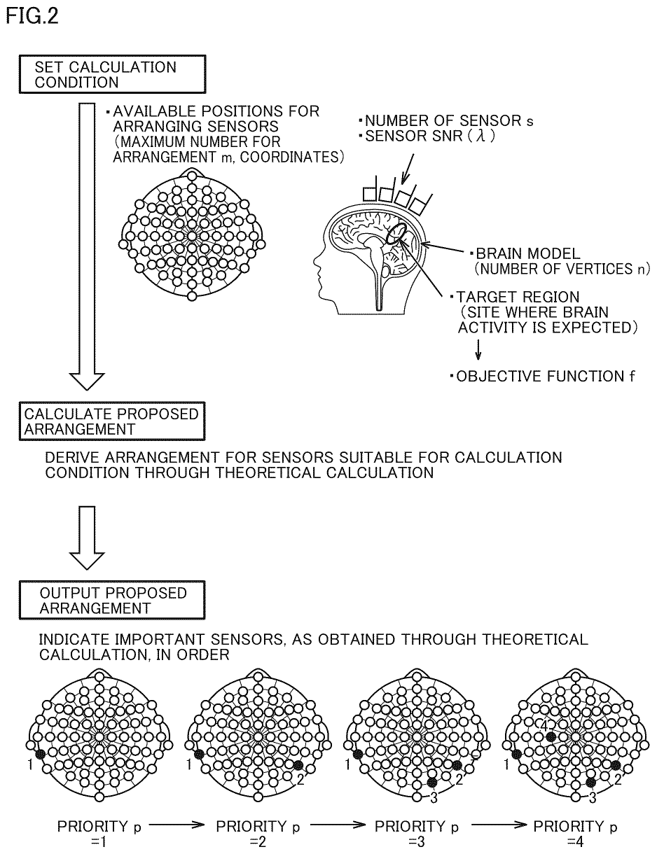

FIG. 2 is a diagram representing a concept of a method for proposing an arrangement for OPM sensors 2 through the arrangement proposing process. In the arrangement proposing process, the steps of setting a calculation condition, calculating a proposed arrangement, and outputting the proposed arrangement are performed in this order.

### [Setting Calculation Condition]

In the step of setting a calculation condition, a calculation condition to be used in calculating a proposed arrangement is set based on input information input to input device 3 and information stored in storage device 14. The calculation condition includes an “available position for arranging a sensor”, a “number of sensors s”, a “sensor SNR (signal-to-noise ratio)”, a “brain model”, a “target region”, an “objective function f”, etc.

An “available position for arranging a sensor” is data indicating an available position for arranging OPM sensor 2 on a surface of the head of a subject. Specifically, an “available position for arranging a sensor” includes a maximum number of OPM sensors 2 that may be arranged (hereinafter also referred to as a “maximum number for arrangement m”), and coordinates of positions at which OPM sensors may be arranged. The coordinates of an available position for arranging a sensor can be determined, for example, based on image data obtained from the head of a subject through MRI or a three-dimensional scanner.

Although FIG. 2 represents the coordinates of an available position for arranging a sensor by a discrete value by way of example, the coordinates of the available position for arranging the sensor can also be handled as a continuous value by determining coordinates on a surface of the head in the form of an equation of a sphere or a plane. Furthermore, the coordinates of an available position for arranging a sensor may be a virtual position at which OPM sensor 2 cannot be arranged in reality.

The “number of sensors s” is the number of OPM sensors 2 actually disposed on the surface of the head of the subject. The number of sensor s is a value equal to or smaller than the maximum number for arrangement m. In the example shown in FIG. 2, the number of sensors s is “4”.

A “sensor SNR” is an SNR of OPM sensor 2. Note that a “sensor SNR” is used to determine a “constant 2” used in calculating an arrangement, as will be described hereinafter.

A “brain model” is obtained by modeling a structure of the brain of a subject using a triangular mesh or the like. The brain model may be generated based on a result of measuring the brain of the subject by MRI, or may be substituted by an existing standard brain model as the subject's brain is assumed to be a standard brain.

A “target region” is a site in the brain model where a biological activity is expected. In calculating a proposed arrangement, as will be described hereinafter, a vertex within a target region in the brain model will be handled as the location of a current source (or a current dipole). A target region may include only one vertex or may include a plurality of vertices.

The “objective function f” is a function used to calculate a proposed arrangement, as will be described hereinafter. As will be described hereinafter, the “objective function f” is determined in accordance with the “target region”.

While in the present embodiment the “objective function f” is set to be a function to maximize a diagonal sum (a sum of diagonal components) of vertices in a target region, this is not exclusive and each function indicated in Olaf Hauk et. al., “Towards an objective evaluation of EEGMEG source estimation methods—The linear approach (Olaf Hauk),” Table 1 may be used as the objective function.

### [Calculating Proposed Arrangement]

In the step of calculating a proposed arrangement, an arrangement for sensors suitable for a set calculation condition is derived through a theoretical calculation. In the present embodiment, each proposed arrangement is assigned a “priority p” indicating an order of importance. How a proposed arrangement is calculated will more specifically be described hereinafter.

### [Outputting Proposed Arrangement]

In the step of outputting a proposed arrangement, data of a proposed arrangement obtained through the theoretical calculation is output. For example, when the data of the proposed arrangement is output to output device 4, output device 4 displays the proposed arrangement.

In the example shown in FIG. 2, a proposed arrangement is displayed for each priority Specifically, the most important arrangement is indicated as a “priority p=1”, and the arrangement of the priority 1 is displayed thereabove. The second most important arrangement is indicated as a “priority p=2”, and the arrangements of the priorities 1 and 2 are displayed thereabove. The third most important arrangement is indicated as a “priority p=3”, and the arrangements of the priorities 1 to 3 are displayed thereabove. The fourth most important arrangement is indicated as a “priority p=4”, and the arrangements of the priorities 1 to 4 are displayed thereabove.

### [Procedure of Arrangement Proposing Process]

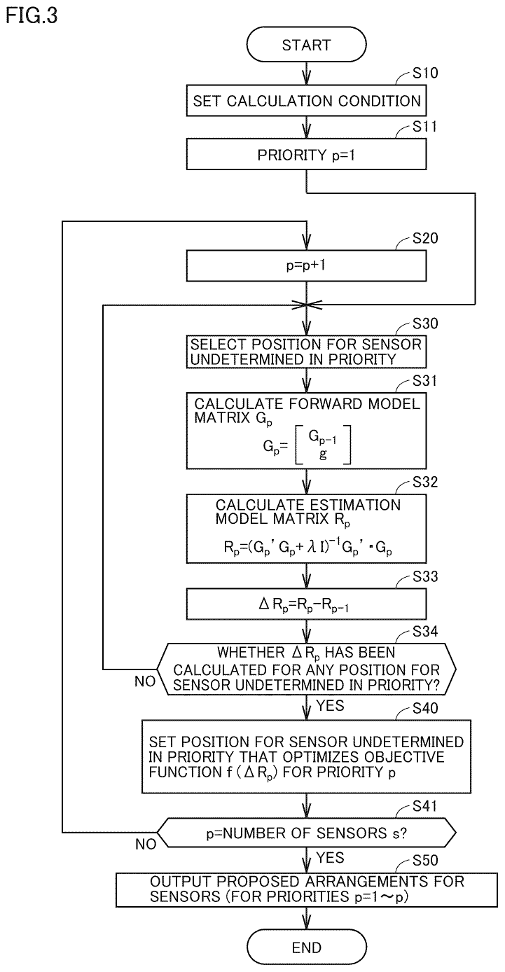

FIG. 3 is a flowchart showing an example of a procedure of the above-described arrangement proposing process.

Initially, computing device 15 sets a calculation condition based on input information input to input device 3 and information stored in storage device 14 (step S10). As described above, the calculation condition includes an available position for arranging a sensor (a maximum number for arrangement m, and coordinates of each arrangement), a number of sensors s, a sensor SNR, a brain model, a target region, an objective function f, and so on.

Subsequently, computing device 15 sets the priority p to an initial value “1” (step S11). Thereafter, computing device 15 proceeds to step S30.

Subsequently, computing device 15 selects any of a plurality of available positions for arranging sensors that is undetermined in priority (step S30).

Subsequently, computing device 15 calculates a forward model matrix G (step S31). The forward model matrix G is a matrix indicating a forward model solution expressing a correspondence between a current dipole moment of a current source and a magnetic field generated at a position of OPM sensor 2 (a magnetic field measured by OPM sensor 2) when the current dipole moment is given to a vertex of the brain model to be the current source. That is, the forward model matrix G satisfies the following equation (1):

[Math. 1]

B=GJ (1)

In equation (1), “B” is a magnetic field vector generated at the position of OPM sensor 2, “J” is a density vector of a true current of a current source in the brain, and “G” is a forward model matrix (a matrix indicating a forward model solution). The forward model matrix G is a matrix that converts the current of the current source to the magnetic field generated at the position of OPM sensor 2, and is also referred to as a “lead field matrix” in the field of magnetoencephalography. The forward model matrix G corresponds to an example of a “forward model” of the present disclosure.

Computing device 15 calculates a forward model matrix Gₚ for a priority p using the following equation (2):

$$\begin{matrix}[ Math . 2 ] & \\ {G}_{p} = [ \begin{matrix}{G}_{p - 1} \\ g\end{matrix} ] & ( 2 )\end{matrix}$$

Specifically, computing device 15 calculates as the forward model matrix Gₚ for the priority p a matrix obtained by adding to a forward model matrix Gₚ₋₁ for an immediately previous priority p−1 a forward model matrix g corresponding to a single sensor selected in step S30 that is undetermined in priority.

The forward model matrix Gₚ has a matrix size of (a priority p)×(a number of vertices v). Therefore, a forward model matrix Gₛ for a priority p having reached a number of sensors s will be (the number of sensors s)×(the number of vertices v). A number of vertices v is a number of vertices of the brain model (a number of points at which a current dipole simulating a neural activity is placed). When a position for priority p=1 is determined, there still is no position for a sensor that is determined in priority, and a forward model matrix G₀ will be an empty matrix. Therefore, a forward model matrix G₁ for the priority 1 will be G₁=g.

Subsequently, computing device 15 uses the forward model matrix G that is calculated in step S31 to calculate an estimation model matrix R (step S32). The estimation model matrix R is a matrix corresponding to an estimation model for estimating the location of a current source in the brain from a result of detection by OPM sensor 2 arranged on the surface of the head. The estimation model matrix R satisfies the following equation (3):

[Math. 3]

J′=RJ (3)

In equation (3), “J” is a density vector of a true current of a current source in the brain, “J” is a density vector of an estimated current of the current source in the brain, and “R” is an estimation model matrix (a matrix indicating an estimated model solution). The estimation model matrix R is a matrix that converts a true current of a current source (or a true value) to a magnetic field generated at a position of a sensor, and reconverts the magnetic field to an estimated current of the current source (or an estimated solution). The estimation model matrix R is also referred to as a “resolution matrix” in the field of magnetoencephalography. The estimation model matrix R corresponds to an example of an “estimation model” of the present disclosure.

Computing device 15 calculates an estimation model matrix Rₚ for the priority p using the following equation (4):

[Math. 4]

Rₚ=(Gₚ′Gₚ+λI)⁻¹Gₚ′·Gₚ (4)

In equation (4), “Gₚ” is a transposed matrix of the forward model matrix Gₚ, “λ” is a constant determined depending on the sensor SNR, and “I” is a unit matrix. The estimation model matrix Rₚ has a matrix size of (the number of vertices v)×(the number of vertices v).

Subsequently, computing device 15 calculates a difference matrix ΔRₚ using the following equation (5) (step S33):

[Math. 5]

ΔRₚ=Rₚ−Rₚ₋₁ (5)

In equation (5), “Rₚ” is the estimation model matrix R for the priority p, and “Rₚ₋₁” is an estimation model matrix R for the priority p−1.

Subsequently, computing device 15 determines whether the difference matrix ΔRₚ has been calculated for any position for a sensor undetermined in priority (step S34).

If the difference matrix ΔRₚ has not been so calculated (NO in step S34), computing device 15 returns to step S30 and repeats steps S30 to S34 until the difference matrix ΔRₚ is calculated for any position for a sensor undetermined in priority.

When the difference matrix ΔRₚ is calculated for any position for a sensor undetermined in priority (YES in step S34), computing device 15 determines any position for a sensor undetermined in priority that optimizes an objective function f (ΔRₚ) based on the difference matrix ΔRₚ, and sets the determined position for the sensor as a position for the priority p (step S40). As will be described hereinafter, the objective function f is determined depending on the location of the target region.

Subsequently, computing device 15 determines whether the priority p has reached a number of sensors s (step S41). If the priority p has not reached the number of sensors s (NO in step S41), computing device 15 moves to step S20 to increment the priority p by 1, and thereafter repeats steps S30 to S34. Steps S30 to S34 are repeated until the priority p reaches the number of sensors s.

By such a repeating process, arrangements for sensors for priorities p=1 to s are sequentially determined in descending order of priority.

For example, let us assume that the maximum number for arrangement m is “10” and the number of sensors s is “3”. In that case, initially, a position for a priority p=1 is determined, then a position for a priority p=2 is determined, and finally a position for a priority p=3 is determined.

At the stage of determining a position for the priority p=1, available positions for arrangement at the ten locations are all undetermined in priority, and accordingly, the forward model matrix G₁, estimation model matrix R₁, and difference matrix ΔR₁ for determining a position for the priority p=1 are each calculated for all of the available positions for arrangement at the ten locations. Then, a position that optimizes the objective function f based on the difference matrix ΔR₁ is set as the position for the priority p=1.

Once the position for the priority p=1 has been set, a process for determining a position for the subsequent priority p=2 is performed. At this stage, a forward model matrix G₂, an estimation model matrix R₂, and a difference matrix ΔR₂ are each calculated for the available positions for arrangement at the remaining nine locations separately, excluding the position for the priority p=1. When this is done, G₂, R₂, and ΔR₂ are calculated using G₁, R₁, and ΔR₁ used to determine the position for the immediately previous priority p=1. Therefore, G₂, R₂, and ΔR₂ will be values reflecting an effect made when a sensor is arranged at the position for the priority p=1. A position that optimizes the objective function f based on ΔR₂ is set as the position for the priority p=2. Therefore, the position for the priority p=2 will be set while the position for the immediately previous priority p=1 is considered.

Once the position for the priority p=2 has been set, a process for determining a position for the last priority p=3 is performed. At this stage, a forward model matrix G₃, an estimation model matrix R₃, and a difference matrix ΔR₃ are each calculated for the available positions for arrangement at the remaining eight locations separately, excluding the positions for the priorities p=1 and 2. When this is done, G₃, R₃, and ΔR₃ are calculated using G₂, R₂, and ΔR₂ for the immediately previous priority p=2. Furthermore, G₂, R₂, and ΔR₂ are calculated using G₁, R₁, and ΔR₁ for the immediately previous priority p=1. Therefore, G₃, R₃, and ΔR₃ will be values reflecting an effect of the positions for the priorities p=1 and 2. A position that optimizes the objective function f based on ΔR₃ is set as the position for the priority p=3. Therefore, the position for the priority p=3 will be set while the positions for the immediately previous priorities p=1 and 2 are considered.

Once the priority p has reached the number of sensors s (YES in step S41), computing device 15 outputs arrangements for sensors for the priorities p=1 to s as a proposed arrangement (step S50). For example, it causes output device 4 to indicate a proposed arrangement for each of the priorities p=1 to s.

Through the above-described arrangement proposing process, processing device 10 can provide a user with a proposed arrangement for s OPM sensors 2 (an arrangement for sensors suitable for estimating the location of a current source in the brain of a subject). The user can accurately estimate the location of the current source by arranging the s OPM sensors 2 in accordance with the proposed arrangement indicated by output device 4. When the proposed arrangement includes a virtual position at which OPM sensor 2 cannot be arranged in reality, OPM sensor 2 may be arranged at a position closest to the virtual position and allowing OPM sensor 2 to be actually arranged thereon.

### [Calculating Forward Model Matrix G]

Hereinafter, a method for calculating the forward model matrix G will be described in detail. As described above, the forward model matrix G is a matrix that satisfies equation (1), i.e., “B=GJ”. Specifically, the forward model matrix G is a matrix that converts a current density vector “J” of a current source in a brain to a magnetic field vector “B” generated at a position of OPM sensor 2.

A relationship between a current generated by the current source in the brain and the magnetic field generated at the position of OPM sensor 2 can be represented using the Biot-Savart law, as indicated by the following equation (6):

$$\begin{matrix}[ Math . 6 ] & \\ B ( r ) = \frac{{μ}_{0}}{4 π} \int J ( {r}^{′} ) \times \frac{r - {r}^{′}}{{\vert r - {r}^{′} \vert}^{3}} {dr}^{′} & ( 6 )\end{matrix}$$

In equation (6), “B” is a magnetic field vector generated at a position of OPM sensor 2, “r” is the position (or coordinates) of OPM sensor 2, “J” is a current density vector of a current source in a brain, and “r” is the location (or coordinates) of the current source in the brain.

The forward model matrix G has components, which can be determined by calculating the equation (6) indicated above. “J” is a current density vector and unless its directionality is specified an enormous amount of expressions will be calculated therefor, however, in the field of current source estimation, it is believed to be reasonable to assume that a current flows in a direction perpendicular to a brain model, and therefore, “J” can be uniquely determined. The position “r” of OPM sensor 2 can be determined by an available position set for a calculation condition for arranging a sensor. The location “r” of the current source can be determined by a position of a vertex of the brain model set for a calculation condition. Therefore, calculating the Biot-Savart equation by the maximum number for arrangement m for sensors per single current source (or vertex) can calculate components/elements of the forward model matrix G for the single current source.

Specifically, the Biot-Savart's law can be written by the maximum number for arrangement m for sensors per current source, as indicated by the following equation (7):

$$\begin{matrix}[ Math . 7 ] & \\ \begin{matrix}Sensor 1 : B ( {r}_{1} ) = \frac{{μ}_{0}}{4 π} \int J ( {r}^{′} ) \times \frac{{r}_{1} - {r}^{′}}{{\vert {r}_{2} - {r}^{′} \vert}^{3}} {dr}^{′} \\ Sensor 2 : B ( {r}_{2} ) = \frac{{μ}_{0}}{4 π} \int J ( {r}^{′} ) \times \frac{{r}_{2} - {r}^{′}}{{\vert {r}_{2} - {r}^{′} \vert}^{3}} {dr}^{′} \\ ⋮ \\ Sensor m : B ( {r}_{m} ) = \frac{{μ}_{0}}{4 π} \int J ( {r}^{′} ) \times \frac{{r}_{m} - {r}^{′}}{{\vert {r}_{m} - {r}^{′} \vert}^{3}} {dr}^{′}\end{matrix} \} & ( 7 )\end{matrix}$$

When the magnitude of a current of the current source is set as a unit current, a magnetic field B generated at a position of OPM sensor 2 per unit current can be determined.

Furthermore, to facilitate understanding, let us consider that OPM sensor 2 has sensitivity only in one axial direction. In that case, “B=GJ” indicated in the equation (1) is expressed by the following equation (8) when each matrix has components/elements visualized.

$$\begin{matrix}[ Math . 8 ] & \\ {\underbrace{( \begin{matrix}{b}_{1} \\ {b}_{2} \\ ⋮ \\ ⋮ \\ ⋮ \\ ⋮ \\ ⋮ \\ ⋮ \\ {b}_{m}\end{matrix} )}}_{B} = {\underbrace{\begin{matrix}( \begin{matrix}{g}_{11} & {g}_{12} & {g}_{13} & … & … & … & … & … & {g}_{1 v} \\ {g}_{21} & {g}_{22} & & & & & & & ⋮ \\ {g}_{31} & & ⋱ & & & & & & ⋮ \\ ⋮ & & & ⋱ & & & & & ⋮ \\ ⋮ & & & & ⋱ & & & & ⋮ \\ ⋮ & & & & & ⋱ & & & ⋮ \\ ⋮ & & & & & & ⋱ & & ⋮ \\ ⋮ & & & & & & & ⋱ & ⋮ \\ {g}_{m 1} & … & … & … & … & … & … & … & {g}_{mv}\end{matrix} ) & \end{matrix}}}_{G} {\underbrace{( \begin{matrix}j \\ {j}_{2} \\ ⋮ \\ ⋮ \\ ⋮ \\ ⋮ \\ ⋮ \\ ⋮ \\ {j}_{v}\end{matrix} )}}_{J} & ( 8 )\end{matrix}$$

Computing device 15 can calculate components/elements of the forward model matrix G by the above-described approach.

[Relationship Between Target Region and Objective Function f]

A “target region” set for a calculation condition is used for setting the “objective function f”. Hereinafter, a relationship between the target region and the objective function f will be described in detail. Note that while the objective function f is a function based on the difference matrix ΔR, as described above, the difference matrix ΔR is a difference of the estimation model matrix R, and, in order to facilitate understanding, the objective function f will be described as being assumed to be based on the estimation model matrix R.

As described above, the estimation model matrix R is a matrix that satisfies equation (3), i.e., J′=RJ. Specifically, the estimation model matrix R is a matrix that converts a true current J to an estimated current J′.

Each row and column of equation (3) have components/elements, which are visualized, as indicated below by the following equation (9):

$$\begin{matrix}[ Math . 9 ] & \\ {\underbrace{( \begin{matrix}{j}_{1}^{′} \\ {j}_{2}^{′} \\ ⋮ \\ ⋮ \\ ⋮ \\ ⋮ \\ ⋮ \\ ⋮ \\ {j}_{v}^{′}\end{matrix} )}}_{{J}^{′}} = {\underbrace{\begin{matrix}( \begin{matrix}{r}_{11} & {r}_{12} & {r}_{13} & … & … & … & … & … & {r}_{1 v} \\ {r}_{21} & {r}_{22} & {r}_{23} & & & & & & ⋮ \\ {r}_{31} & {r}_{32} & {r}_{33} & & & & & & ⋮ \\ ⋮ & & & {r}_{44} & & & & & ⋮ \\ ⋮ & & & & {r}_{55} & & & & ⋮ \\ ⋮ & & & & & {r}_{56} & & & ⋮ \\ ⋮ & & & & & & ⋱ & & ⋮ \\ ⋮ & & & & & & & ⋱ & ⋮ \\ {r}_{v 1} & … & … & … & … & … & … & … & {r}_{vv}\end{matrix} ) & \end{matrix}}}_{R} {\underbrace{( \begin{matrix}j \\ {j}_{2} \\ ⋮ \\ ⋮ \\ ⋮ \\ ⋮ \\ ⋮ \\ ⋮ \\ {j}_{v}\end{matrix} )}}_{J} & ( 9 )\end{matrix}$$

In equation (9), “j₁ to jₙ” are true currents at each vertex of a brain model, and “j₁′ to jₙ” are estimated currents at each vertex of the brain model.

When a target region is a region including four vertices (vertices 1, 2, 3, 4) of a brain model, the estimation model matrix R will have the first to fourth rows and the first to fourth columns to be matrix elements relevant to the target region, as indicated by the following equation (10):

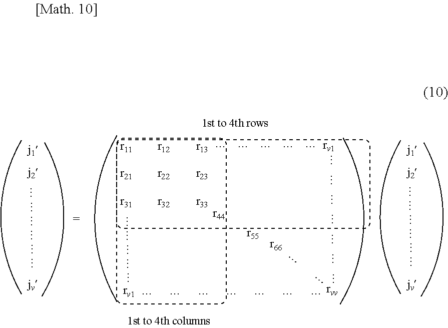

When the estimation model matrix R is viewed in detail, an effect that the true current at the vertex 1 has on currents estimated for vertices 1 to v and an effect that the true currents at the vertices 1 to v have on a current estimated for the vertex 1 can be read. When this is considered, one ideal form of the estimation model matrix R may be considered as a matrix having a value only at a diagonal component at which a true value is present. That is, the objective function f can be set to be a function that maximizes a diagonal sum (a sum of diagonal components) of vertices in a target region. In view of this, computing device 15 determines the objective function f depending on the location of the target region (more specifically, of a vertex within the target region).

When a target region to be optimized is widened and includes vertices 1 to 4 therein, a function that maximizes a diagonal sum of components r11 to r44 of the estimation model matrix R can be set for the objective function f as indicated below by the following equation (11). When a region in which a brain activity occurs can be predicted, and it is not narrowed down to a specific site, a wide target region is assumed as described above.

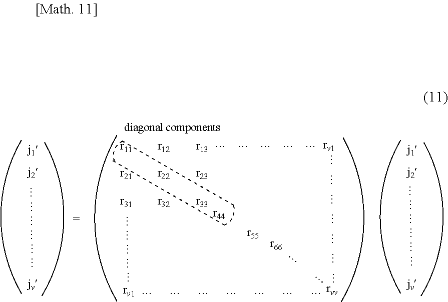

In contrast, when a target region to be optimized is narrowed and includes the vertex 1 alone, a function that maximizes the diagonal component r11 of the estimation model matrix R can be set for the objective function f. When a region in which a brain activity occurs can be pinpointed and thus predicted, a narrow target region is assumed in this manner.

Furthermore, the objective function f may be set to be a function that maximizes a ratio between a diagonal sum (a sum of diagonal components) of a target region and a non-diagonal sum (a sum of non-diagonal components) of the target region, as indicated below by an equation (12). Alternatively, the objective function f may be set to be a function that maximizes a ratio between a diagonal sum of a target region and the other elements thereof.

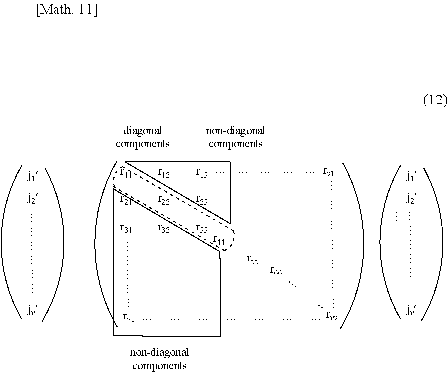

As described above, computing device 15 according to the present embodiment calculates the forward model matrix G and the estimation model matrix R based on a calculation condition including a target region and an available position for arranging a sensor, etc. Then, computing device 15 sets and outputs a proposed arrangement (or priority p) for OPM sensor 2 with reference to an indication that the objective function f set based on the estimation model matrix R is optimized or the like. The user can efficiently perform highly sensitive magnetoencephalography by arranging a plurality of OPM sensors 2 in accordance with the proposed arrangement.

The arrangement proposing method of the present embodiment can propose an arrangement for a plurality of OPM sensors 2 that is suitable for current source estimation. This can efficiently perform optimization of arrangement for sensors that has conventionally been difficult with SQUIDs. Furthermore, it allows magnetoencephalography with high sensitivity (or high accuracy). Furthermore, it can also reduce the number of sensors necessary for current source estimation, and hence hardware cost for a magnetoencephalography system.

### Second Embodiment

In a second embodiment, magnetoencephalographic measurement is divided and thus performed twice, as described hereinafter, in order to perform magnetoencephalographic measurement of higher accuracy efficiently with a limited number of OPM sensors 2.

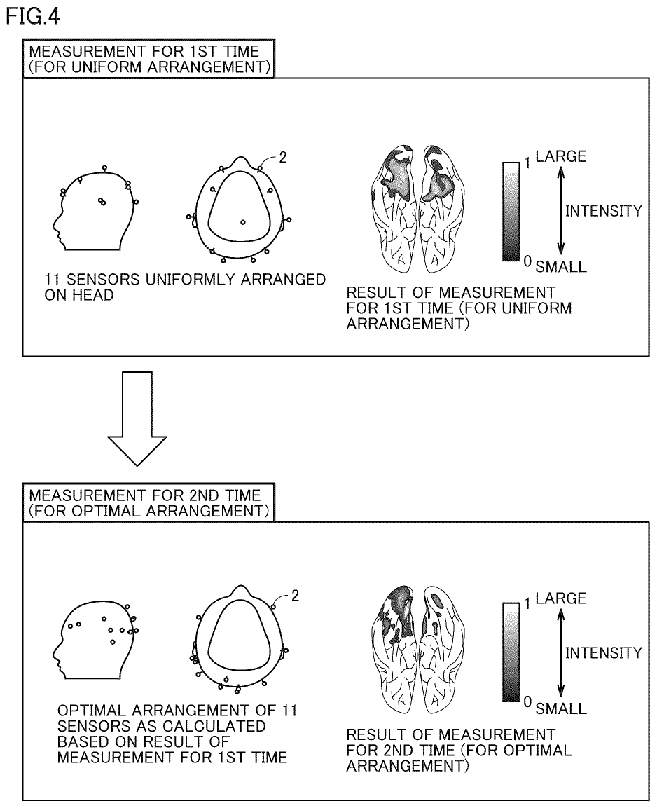

FIG. 4 is a diagram showing an example of an arrangement for sensors when 11 OPM sensors 2 are used to perform magnetoencephalographic measurement twice, as well as a result of the measurement.

For a measurement for a first time, it is unknown where in the brain a current source (a true abnormal region) exists, and accordingly, the 11 OPM sensors 2 are uniformly arranged on the surface of the head with the entire brain considered as a region to be inspected. Then, from results of measurements by the 11 uniformly arranged OPM sensors 2, a location for the current source is provisionally measured. In the example shown in FIG. 4, a result of the measurement for the first time indicates that more measurement results stronger in intensity are observed in the left brain than in the right brain, and the left brain is provisionally measured as a provisional location for the current source. Note that a portion of the left brain where intensity of a predetermined value or more concentrates may be set as the provisional location for the current source.

For a measurement for a second time, the provisional location of the current source provisionally measured in the measurement for the first time (i.e., the left brain) is set as a “target region” and the above-described arrangement proposing process is thus performed, and the 11 OPM sensors 2 are rearranged in accordance with a proposed arrangement obtained through the arrangement proposing process. And from a result of measurements by the rearranged eleven OPM sensors 2, a location for the current source is formally measured. In the measurement for the second time, OPM sensors 2 can be rearranged at optimal positions based on a result of the measurement for the first time, and a measurement result of maximum intensity is seen at a position closer to a true value (a true abnormal region). That is, in the measurement for the second time, the current source is estimated with higher accuracy.

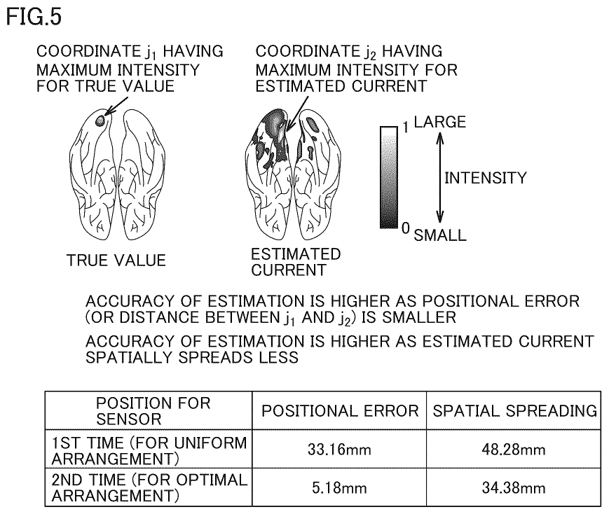

FIG. 5 is a diagram comparing in accuracy the measurement for the first time (or for the uniform arrangement) and the measurement for the second time (or for the optimal arrangement) shown in FIG. 4. It can be said that a current source is estimated with higher accuracy when there is a smaller positional error between a true value and an estimated value (that is, a smaller shortest distance between a coordinate j₁ having maximum intensity for the true value and a coordinate j₂ having maximum intensity for an estimated current) and when the estimated current spatially spreads less (or the estimated current is measured in a region having a smaller maximum length).

As shown in FIG. 5, it can be understood that in the measurement for the first time (for the uniform arrangement) the true value and the estimated value have a positional error of 33.16 mm therebetween and the estimated current has a spatial spreading of 48.28 mm, whereas in the measurement for the second time (for the optimal arrangement) the true value and the estimated value have an improved positional error of 5.18 mm therebetween and the estimated current has an improved spatial spreading of 34.38 mm. In particular, the positional error between the true value and the estimated value is significantly improved (by about 28 mm), and the estimated current's maximum intensity is only about 5 mm deviating from the true value (an abnormal region that is a true activity source) by the optimal arrangement.

As described above, according to the second embodiment, in the measurement for the first time, a plurality of OPM sensors 2 are uniformly arranged on a surface of a head to provisionally measure a location for a current source, the location of the provisionally measured current source is set in a target region to perform an arrangement proposing process, and the plurality of OPM sensors 2 are rearranged according to a proposed arrangement obtained through the arrangement proposing process to measure a location for the current source for a second time. Thus, magnetoencephalographic measurement of high accuracy can efficiently be performed with a limited number of OPM sensors 2.

The first provisional measurement is not limited to using OPM sensor 2 insofar as a provisional location for a current source can be measured. For example, in the first provisional measurement, fMRI (functional Magnetic Resonance Imaging) or the like may be employed to measure a provisional location for the current source. Furthermore, a provisional location for the current source may be determined via a medical practitioner's findings.

### [First Modification]

For example, in FIG. 3 at step S50 as described above, in addition to causing output device 4 to display a proposed arrangement for each priority, output device 4 may be caused to display a positional error of each proposed arrangement between a target region and a result of current source estimation (a positional error between a true current and an estimated current).

This allows the user to look at output device 4 to confirm whether the positional error of the proposed arrangement for each priority is within an allowable range, and thereafter determine a number of sensors s to be used for current source estimation.

This can also reduce the number of sensors s used for current source estimation, as required, and hence cost for hardware for a magnetoencephalography system.

### [Second Modification]

The presently disclosed arrangement proposing method is applicable to a sensor other than OPM sensor 2. The presently disclosed arrangement proposing method is applicable for example to a fluxgate sensor, an MR (magneto resistance) sensor, an MI (magneto impedance) sensor, a coil type sensor, or an NVC (nitrogen-vacancy center in diamond) sensor in place of or in addition to OPM sensor 2.

### [Third Modification]

The presently disclosed arrangement proposing method is also applicable to fields other than magnetoencephalography. The presently disclosed arrangement proposing method is also applicable to a field of measuring a magnetic field of a living body other than the brain (e.g., magnetic fields generated by a biological activity in the heart, the spinal cord, peripheral nerves or muscles, etc.).

### [Fourth Modification]

The presently disclosed arrangement proposing method is applicable to a sensor other than a magnetic sensor. That is, the presently disclosed arrangement proposing method is also applicable to an electroencephalography (EEG) sensor, an electromyography (EMG) sensor, or a similar potential sensor that detects a potential generated by an electrical activity of a living body.

### [Fifth Modification]

The presently disclosed arrangement proposing method can also be applied to a SQUID difficult to positionally change. As described above, the SQUID is disposed in a dewar filled with liquid helium and it is difficult to easily change its arrangement. However, it is possible to arrange a large number (e.g., several tens or several hundreds) of SQUIDs on the head of a subject in advance. The above-described arrangement proposing method can be applied to a process for proposing a position for a SQUID of a plurality of SQUIDs previously arranged on the head of the subject, that is useful for current source estimation.

Specifically, this may be done by arranging a plurality of SQUIDs in advance on the head of the subject, setting a calculation condition including a location of a target region and an arrangement for the plurality of SQUIDs, calculating priority based on the calculation condition for all or some of the plurality of SQUIDs, and estimating the location of a current source in the brain of the subject using a result of detection by an electromagnetic sensor selected based on the priority.

Thus, out of a large number of SQUIDs arranged in advance on the head of the subject, a SQUID having a high priority can be selected, and a result of measurement by the selected SQUID can be used to perform current source estimation. Therefore, even when a SQUID difficult to positionally change is used, arranging a large number of electromagnetic sensors in advance on the head of the subject allows current source estimation to be performed accurately.

### [Aspects]

It is understood by those skilled in the art that the above-described embodiments and modifications thereof are specific examples of the following aspects.

(Clause 1) According to one aspect, a method for proposing an arrangement for an electromagnetic sensor is a method for proposing an arrangement for a plurality of electromagnetic sensors on a biological surface around a target region inside a subject, the method using a forward model for estimating an electromagnetic field generated at positions of the plurality of electromagnetic sensors by a biological activity caused inside the subject, the method comprising: setting a calculation condition including a location of the target region and available positions for arranging the plurality of electromagnetic sensors; calculating a proposed arrangement based on the set calculation condition for all or some of the plurality of electromagnetic sensors for estimating a location of a current source inside the subject; and outputting the proposed arrangement.

According to the arrangement proposing method described in clause 1, a proposed arrangement for sensors is calculated based on a calculation condition, and output. This can provide a user with a proposed arrangement for sensors suitable for current source estimation. The user can accurately estimate the location of a current source by arranging a plurality of electromagnetic sensors according to the proposed arrangement.

(Clause 2) The method according to clause 1, wherein the calculating a proposed arrangement comprises calculating priority for all or some of the available positions.

According to the arrangement proposing method described in clause 2, priorities of all or some of the available positions can be provided to the user.

(Clause 3) The method according to clause 1 or 2, further comprising provisionally measuring the location of the current source. The setting a calculation condition comprises setting the provisionally measured location of the current source as the location of the target region.

According to the arrangement proposing method described in clause 3, the location of the current source is provisionally measured before the proposed arrangement is calculated, and the proposed arrangement is calculated based on the provisionally measured location of the current source. The proposed arrangement can thus be efficiently calculated with high accuracy.

(Clause 4) The method according to any one of clauses 1 to 3, wherein the setting a calculation condition comprises setting a parameter of the forward model as the calculation condition.

According to the arrangement proposing method described in clause 4, the proposed arrangement can be calculated by setting a parameter of the forward model as the calculation condition.

(Clause 5) The method according to any one of clauses 1 to 4, wherein the calculating a proposed arrangement comprises calculating the proposed arrangement using an estimation model, the estimation model estimating the location of the current source from a result of detection by all or some of the plurality of electromagnetic sensors. The calculating a proposed arrangement comprises calculating the proposed arrangement to reduce a difference between a result of an estimation by the estimation model as determined by setting a parameter of the estimation model as the calculation condition and the location of the target region.

According to the arrangement proposing method described in clause 5, the proposed arrangement can be calculated to reduce a difference between a result of an estimation by the estimation model (the location of an estimated current) and the location of the target region (the location of a true current).

(Clause 6) The method according to clause 5, wherein the outputting the proposed arrangement comprises presenting to a user the difference between the result of the estimation by the estimation model and the location of the target region in the proposed arrangement.

According to the arrangement proposing method described in clause 6, the user can confirm the difference between the result of the estimation by the estimation model and the location of the target region as a level in accuracy of current source estimation through the proposed arrangement.

(Clause 7) The method according to any one of clauses 1 to 6, wherein the calculation condition includes, in addition to the location of the target region and the available positions, at least one of: a number of the plurality of electromagnetic sensors; and a signal to noise ratio (SNR) of the plurality of electromagnetic sensors.

According to the arrangement proposing method described in clause 7, the proposed arrangement can be calculated while at least one of the number of electromagnetic sensors and the SNR thereof is considered.

(Clause 8) The method according to any one of clauses 1 to 7, wherein the electromagnetic sensor is an OPM (optically pumped magnetometer) sensor, a fluxgate sensor, an MR (magneto resistance) sensor, an MI (magneto impedance) sensor, a coil type sensor, or an NVC (nitrogen-vacancy center in diamond) sensor.

According to the arrangement proposing method described in clause 8, a proposed arrangement for an OPM sensor, a fluxgate sensor, an MR sensor, an MI sensor, a coil type sensor or an NVC sensor can be provided to the user. (Clause 9) The method according to any one of clauses 1 to 8, wherein the electromagnetic sensor is a magnetic sensor or a potential sensor that detects a magnetic field generated by an electrical activity in a brain, a heart, a spinal cord, peripheral nerves, or muscles.

According to the arrangement proposing method described in clause 9, a proposed arrangement for a magnetic sensor or a potential sensor that detects a magnetic field generated by an electrical activity in the brain, the heart, the spinal cord, peripheral nerves, or muscles can be provided to the user.

(Clause 10) The method according to any one of clauses 1 to 9, wherein the outputting the proposed arrangement comprises presenting the proposed arrangement to a user.

According to the arrangement proposing method described in clause 10, the proposed arrangement can be presented to the user.

(Clause 11) According to one aspect, a method for estimating a location of a current source is a method for estimating a location of a current source inside a subject by using a result of detection by a plurality of electromagnetic sensors each disposed on a biological surface of the subject to detect an electromagnetic field generated by a biological activity caused in a target region inside the subject, the method comprising: setting a calculation condition including a location of the target region and an arrangement for the plurality of electromagnetic sensors; calculating priority based on the calculation condition for all or some of the plurality of electromagnetic sensors; and estimating the location of the current source using a result of detection by an electromagnetic sensor selected based on the priority.

According to the arrangement proposing method described in clause 11, current source estimation is performed using a result of detection by an electromagnetic sensor having a high priority among a large number of electromagnetic sensors. Therefore, even when an electromagnetic sensor (SQUID or the like) difficult to positionally change is used, arranging a large number of electromagnetic sensors in advance on a biological surface of the subject allows current source estimation to be accurately performed by using a result of detection by a sensor selected from the large number of electromagnetic sensors as the sensor has higher priority.

(Clause 12) According to one aspect, an apparatus that proposes an arrangement for an electromagnetic sensor is an apparatus that proposes an arrangement for a plurality of electromagnetic sensors on a biological surface around a target region inside a subject, the apparatus using a forward model for estimating an electromagnetic field generated at positions of the plurality of electromagnetic sensors by a biological activity caused inside the subject, the apparatus comprising: an input unit that receives a calculation condition including a location of the target region and available positions for arranging the plurality of electromagnetic sensors; a computing device that calculates a proposed arrangement based on the calculation condition for all or some of the plurality of electromagnetic sensors for estimating a location of a current source inside the subject; and an output unit that outputs the proposed arrangement.

The arrangement proposing method described in clause 12 can achieve a function and effect similar to those of the arrangement proposing method described in clause 1.

It should be understood that the embodiments disclosed herein are illustrative and non-restrictive in any respect. The scope of the present invention is defined by the terms of the claims rather than by the foregoing description of the embodiments, and is intended to encompass any modification within the meaning and scope equivalent to the terms of the claims.

REFERENCE SIGNS LIST

1 data processing system, 2 OPM sensor, 3 input device, 4 output device,
10 processing device, 11 sensor interface, 12 input interface, 13 output
interface, 14 storage device, 15 computing device.

1. A method for proposing an arrangement for a plurality of electromagnetic sensors on a biological surface around a target region inside a subject, the method using a forward model for estimating an electromagnetic field generated at positions of the plurality of electromagnetic sensors by a biological activity caused inside the subject, the method comprising:
setting a calculation condition including a location of the target region and available positions for arranging the plurality of electromagnetic sensors;
calculating a proposed arrangement based on the set calculation condition for all or some of the plurality of electromagnetic sensors for estimating a location of a current source inside the subject; and
outputting the proposed arrangement.

2. The method according to claim 1, wherein the calculating a proposed arrangement comprises calculating priority for all or some of the available positions.

3. The method according to claim 1, further comprising provisionally measuring the location of the current source,
wherein the setting a calculation condition comprises setting the provisionally measured location of the current source as the location of the target region.

4. The method according to claim 1, wherein the setting a calculation condition comprises setting a parameter of the forward model as the calculation condition.

5. The method according to claim 1, wherein
the calculating a proposed arrangement comprises calculating the proposed arrangement using an estimation model, the estimation model estimating the location of the current source from a result of detection by all or some of the plurality of electromagnetic sensors, and
the calculating a proposed arrangement comprises calculating the proposed arrangement to reduce a difference between a result of an estimation by the estimation model as determined by setting a parameter of the estimation model as the calculation condition and the location of the target region.

6. The method according to claim 5, wherein the outputting the proposed arrangement comprises presenting to a user the difference between the result of the estimation by the estimation model and the location of the target region in the proposed arrangement.

7. The method according to claim 1, wherein the calculation condition includes, in addition to the location of the target region and the available positions, at least one of: a number of the plurality of electromagnetic sensors; and a signal to noise ratio (SNR) of the plurality of electromagnetic sensors.

8. The method according to claim 1, wherein the electromagnetic sensor is an OPM (optically pumped magnetometer) sensor, a fluxgate sensor, an MR (magneto resistance) sensor, an MI (magneto impedance) sensor, a coil type sensor, or an NVC (nitrogen-vacancy center in diamond) sensor.

9. The method according to claim 1, wherein the electromagnetic sensor is a magnetic sensor or a potential sensor that detects a magnetic field generated by an electrical activity in a brain, a heart, a spinal cord, peripheral nerves, or muscles.

10. The method according to claim 1, wherein the outputting the proposed arrangement comprises presenting the proposed arrangement to a user.

11. A method for estimating a location of a current source inside a subject by using a result of detection by a plurality of electromagnetic sensors each disposed on a biological surface of the subject to detect an electromagnetic field generated by a biological activity caused in a target region inside the subject, the method comprising:
setting a calculation condition including a location of the target region and an arrangement for the plurality of electromagnetic sensors;
calculating priority based on the calculation condition for all or some of the plurality of electromagnetic sensors; and
estimating the location of the current source using a result of detection by an electromagnetic sensor selected based on the priority.

12. An apparatus that proposes an arrangement for a plurality of electromagnetic sensors on a biological surface around a target region inside a subject, the apparatus using a forward model for estimating an electromagnetic field generated at positions of the plurality of electromagnetic sensors by a biological activity caused inside the subject, the apparatus comprising:
an input unit that receives a calculation condition including a location of the target region and available positions for arranging the plurality of electromagnetic sensors;
a computing device that calculates a proposed arrangement based on the calculation condition for all or some of the plurality of electromagnetic sensors for estimating a location of a current source inside the subject; and
an output unit that outputs the proposed arrangement.
